(19) United States
(12) Patent Application Publication
(10) Pub. No.: US 2024/0122222 A1
(43) Pub. Date: Apr. 18, 2024

(54) METHODS AND SYSTEMS OF PRODUCING NUTRITIONAL SUPPLEMENT FROM MICROBIAL CELLS

(71) Applicant: Solar Foods Oy, Lappeenranta (FI)

(72) Inventors: Dilek ERCILI-CURA, Lappeenranta (FI); Juha-Pekka PITKÄNEN, Lappeenranta (FI)

(73) Assignee: Solar Foods Oy, Lappeenranta (FI)

(21) Appl. No.: 18/554,135

(22) PCT Filed: Apr. 7, 2022
(86) PCT No.: PCT/FI2022/050228

§ 371 Date: Oct. 5, 2023

(30) Foreign Application Priority Data
Apr. 27, 2021 (FI) 20215488

(51) Int. Cl. A23L 33/135; A23L 33/00; C12M 1/00; C12N 1/02; C12N 1/20

(52) U.S. Cl. CPC A23L 33/135; A23L 33/40; C12M 47/02; C12M 47/08; C12M 47/14; C12M 47/20; C12N 1/02; C12N 1/205; C12N 2523/00; C12R 2001/01

(57) ABSTRACT

A method of producing a nutritional supplement from microbial cells. The method includes cultivating microbial cells to obtain a biomass; incubating the biomass with a heat treatment at temperature from 55° C. up to 80° C. for an incubation time from 10 minutes up to 60 minutes; concentrating the biomass by separating and removing a liquid phase from a solid phase to obtain a dry matter content from 2% up to 40% of total weight of the nutritional supplement. Disclosed also is a system of producing a nutritional supplement from microbial cells using the aforementioned method. The system includes a bioreactor; a heat-exchanger and a separator for carrying out the respective steps of the aforementioned method.

Reference is made to the Identification of the Microorganism, with the identification reference given by the Depositor SoF1, having the Accession number given by the INTERNATIONAL DEPOSITORY AUTHORITY, VTT E-193585, received on Jun. 11, 2019

### TECHNICAL FIELD

The present disclosure relates generally to bioprocessing of grown microbial mass; and more specifically to methods and systems of producing nutritional supplement from microbial cells. In this regard, the present disclosure relates to removing endotoxins from food ingredients, particularly a single cell nutritional supplement, during production, particularly downstream processing.

### BACKGROUND

With the current outburst of innovations in food science and technology, alternative and new food concepts, such as alternative food, especially alternative protein, are introduced. In this regard, microbes are grown in bioreactors with minimal use of natural resources and with minimal green-house gas emissions and then converting the biomass into food or feed is a promising sustainable protein production method. Few commercial examples already exist, such as mycoprotein (fungal biomass) in the form of meat alternatives (Quorn®) or various bacterial biomasses used as feed additive (e.g. Pekilo®, Uniprotein). In those cases, microbial biomass is not a contaminant found in minor amounts but comprise a large portion of the food or feed. When those food and feed concepts are prepared using Gram-negative bacteria (such as E. coli and Bacteroidetes) as single source or part of a larger flora, control of endotoxin content becomes highly important.

Notably, the Gram-negative bacteria are a part of our environment and contribute to the gut flora of the humans as a result of ingestion or inhalation thereby. The Gram-negative bacteria have the ability to translocate from the gut into the bloodstream and result in stimulation of the human immune system. Specifically, microbial endotoxins, a type of lipopolysaccharides (LPS), are responsible for the aforesaid stimulation that causes toxic effects. More specifically, endotoxins when released in sufficiently large quantities in blood result in the toxic effects, including fever and possibly fatal septic shocks, apart from low-grade inflammation, liver damage, diabetes, obesity and cognitive disorders associated with low levels of endotoxins in the blood.

The biggest source of endotoxin content in human (or livestock) daily diet is contamination of food with Gram-negative bacteria. Other most common source of ingested endotoxins are probiotic capsules, supplements or other food ingredients. Most of Gram-negative probiotics are of an E. coli strain or belong to the Enterobacteriaceae family. Normally, when a food product is ingested, the epithelial cells of bowels act as a physical barrier with the production of a mucus layer that prevents the bacterial and LPS translocation into the bloodstream. However, in case of endotoxemia or leaky gut syndrome, bacterial translocation can occur due to mucosal degradation. Moreover, there are no endotoxin limits set for those ingested examples as ingested endotoxins does not comprise a health risk, which is also related to low cell density due to such uptake when compared to Gram-negative bacteria levels present in gut (as discussed by Wassenaar and Zimmerman, 2018).

Conventional techniques for preventing endotoxins from entering body include a preliminary testing of any food product or pharmaceutical product that will enter the body for the presence of endotoxins. In this regard, the pharmaceutical product including parenteral medicines and injectable devices are tested before its release into the market. In this regard, advanced assays and sterilization techniques have been employed for respectively estimating endotoxin amounts and destroying the microbial endotoxins. However, microbial endotoxins are highly heat-stable and are not destroyed under regular sterilizing conditions. Conventionally, at the laboratory scale, microbial endotoxins can be inactivated when exposed to—temperature of 250° C. for more than 30 minutes or 180° C. for more than 3 hours, or acids or alkalis of at least 0.1 M strength. Other endotoxin removal methods for protein solutions are usually based on chromatography or detergent phase partitioning including ion-exchange chromatography, affinity adsorbents, such as immobilized L-histidine, poly-L-lysine, poly(methyl L-glutamate), and polymyxin B, gel filtration chromatography, ultrafiltration, sucrose gradient centrifugation, and Triton X114 phase separation, filtration, affinity adsorbent, active carbon, nanoparticle-based methods and others. Notably, multiple cycles are required to be performed to completely remove endotoxins and make products non-pyrogenic. Those processes are utilized in pharmaceutical industry, e.g. production of antibiotics by Gram-negative organisms or elimination of contamination risks in intravenous drugs and devices.

However, food products are considered differently than intravenous pharmaceutical products in terms of endotoxicology (presence of LPS in the blood). Therefore, the aforesaid techniques are not applicable in food production due to high costs associated, potential toxicity introduced by the reagents and also differing purity level requirements in food and pharmaceutical applications. Moreover, there are no processes known used specially in food industry other than elimination of contamination risks of food, which means keeping good production and handling practices in place.

Therefore, in light of the foregoing discussion, there exists a need to overcome drawbacks associated with conventional techniques for producing nutritional supplement from microbial cells while reducing the endotoxins therein.

### SUMMARY

The present disclosure seeks to provide a method of producing nutritional supplement from microbial cells. The present disclosure also seeks to provide a system of producing nutritional supplement from microbial cells. The present disclosure seeks to provide a solution to the existing problem of efficiently reducing endotoxins in the food products, nutraceuticals, and so forth. An aim of the present disclosure is to provide a solution that overcomes at least partially the problems encountered in prior art, and provides an efficient and robust technique for producing nutritional supplement from microbial cells that leads to reduced endotoxin levels in the nutritional supplement, and consequently using the said nutritional supplement for food or feed production in the form of slurry or powder, essentially described as heat-inactivated, dried nutritional supplement.

In an aspect, an embodiment of the present disclosure provides a method of producing a nutritional supplement from microbial cells, the method comprising:
cultivating the microbial cells to obtain a biomass comprising gram-negative bacteria;
incubating the biomass for at least partially degrading walls of the microbial cells with a heat treatment at temperature from 55° C. up to 80° C. for an incubation time from 10 minutes up to 60 minutes;
concentrating the incubated biomass by separating and removing a liquid phase from a solid phase to obtain a dry matter content from 2% up to 40% of total weight of the nutritional supplement: and
homogenizing the nutritional supplement with high-pressure homogenization at least one run for at least partially degrading the walls of the microbial cells.

In another aspect, an embodiment of the present disclosure provides a system of producing a nutritional supplement from microbial cells, the system comprising:
a bioreactor for cultivating the microbial cells to obtain a biomass comprising gram-negative bacteria;
a heat-exchanger having incubation temperature from 55° C. up to 80° C. and incubation time from 10 minutes up to 60 minutes for partially degrading walls of the microbial cells;
a separator for separating a solid phase and a liquid phase to obtain a dry matter content from 2% up to 40% of total weight of the nutritional supplement; and
a high-pressure homogenizer for at least partially degrading the walls of the microbial cells of the nutritional supplement.

Embodiments of the present disclosure substantially eliminate or at least partially address the aforementioned problems in the prior art, and enable providing a bioprocess, comprising cultivating, incubating and separating, for use thereof in food processing readily. Moreover, the selected conditions of the aforesaid bioprocess result in a final nutritional supplement produced from microbial cells and comprising gram-negative bacteria with reduced endotoxin levels without adding significant operational costs to production.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIGS. 1, 2 are flowcharts depicting steps of a method of producing nutritional supplement from microbial cells, in accordance with an embodiment of the present disclosure;

FIG. 3 is an illustration of low-endotoxin bacterial biomass production process, in accordance with an embodiment of the present disclosure;

FIG. 4 is a block diagram of a system of producing nutritional supplement from microbial cells, in accordance with an embodiment of the present disclosure;

FIG. 5 is a block diagram of a system of producing a high-moisture meat analogue, in accordance with an embodiment of the present disclosure;

FIG. 6 is a schematic illustration of a process of producing a high-moisture meat analogue, in accordance with an embodiment of the present disclosure; and

FIGS. 7A, 7B and 8 are the LAL assay results of different samples showing intermediate endotoxic activity, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a method of producing a nutritional supplement from microbial cells, the method comprising:
cultivating the microbial cells to obtain a biomass comprising gram-negative bacteria;
incubating the biomass for at least partially degrading walls of the microbial cells with a heat treatment at temperature from 55° C. up to 80° C. for an incubation time from 10 minutes up to 60 minutes;
concentrating the incubated biomass by separating and removing a liquid phase from a solid phase to obtain a dry matter content from 2% up to 40% of total weight of the nutritional supplement; and
homogenizing the nutritional supplement with high-pressure homogenization at least one run for at least partially degrading the walls of the microbial cells.

In another aspect, an embodiment of the present disclosure provides a system of producing a nutritional supplement from microbial cells, the system comprising:
a bioreactor for cultivating the microbial cells to obtain a biomass comprising gram-negative bacteria;
a heat-exchanger having incubation temperature from 55° C. up to 80° C. and incubation time from 10 minutes up to 60 minutes for partially degrading walls of the microbial cells;
a separator for separating a solid phase and a liquid phase to obtain a dry matter content from 2% up to 40% of total weight of the nutritional supplement; and
a high-pressure homogenizer for at least partially degrading the walls of the microbial cells of the nutritional supplement.

The present disclosure provides the aforementioned method of producing nutritional supplement from microbial cells. The method of the present disclosure comprises growing microbial cell to produce a biomass and treating the said biomass, by means of downstream processing, to produce nutritional supplement for use in food or feed. The treatment process of biomass comprising gram-negative bacteria includes steps of cultivation, incubation and separation to effectively disrupt the cell membrane of the microbial cell and release the microbial endotoxins (the lipopolysaccharides) from the cell membrane. Beneficially, the treatment process renders a concentrated nutritional supplement with significantly reduced amounts of endotoxins for production of feed, food or food ingredients. Additionally, the method of the present disclosure enables utilization of a wide variety of cells including, but not limited to, bacteria, fungi, protozoans, plant cells, animal cells and the like, for growing and treatment thereof for producing nutritional supplement, thereby solving the problem associated with the undeniable need for alternative food, especially alternative protein, in human (and livestock) diet that stem from the fact that our current food system is not sustainable and is a big contributor to environmental damage. Moreover, the selected conditions of the aforesaid method result in a final nutritional supplement produced from microbial cells with reduced endotoxin levels without adding significant operational costs to production.

Throughout the present disclosure, the term “nutritional supplement” as used herein refers to a nutritional supplement extracted from microbial cells. The nutritional supplement provides a concentrated source of proteins with no or negligible carbohydrates, fats or any other compounds. Alternatively, the nutritional supplement produced from microbial cells comprises proteins and are also fortified with compounds such as vitamins and minerals, such as iron. Notably, proteins are building blocks of body and typically essential for muscle building and recovery. It will be appreciated that the protein consumption should be monitored to avoid affecting kidneys, liver and body's bone and calcium balance reported from long-term excessive protein consumption.

Normally, around 0.5 to 2.5 gram of protein per kilogram of body weight should be consumed per day for a healthy body. Optionally, the nutritional supplement can be mixed with water, milk, fruit or vegetable juices or smoothies, and the like for consumption by a human or an animal (including birds, fishes, and the like).

The method of producing nutritional supplement from microbial cells comprises cultivating microbial cells to obtain a biomass. The term “microbial cells” as used herein refers to microorganisms that are rich sources of proteins. The microorganisms typically include bacteria, archaea, fungi, protists, and so forth. The microbial cells are cultured under controlled conditions to build microbial mass. The term “biomass” as used herein refers to a measure of amount of living component (namely, microbes) in a sample, such as a culture media, that grow from an initial amount of microbial cells (inoculum) and continue to reproduce further. It will be appreciated that a single type of microbes or a combination of microbes could be grown to obtain biomass. It will be appreciated that microbes have shorter reproduction time and, thus, can be grown rapidly to produce high cell density microbial mass. Moreover, the microbial cells are a rich source of proteins and, thus may be exploited to produce single cell proteins, i.e. a potential alternative for the animal- or plant-derived protein, for consumption by for example humans. However, the microbial cells have small size and low density, and thus require producing a large amount of microbial mass to meet the protein demand of the consumers. Moreover, harvesting a larger amount of microbial mass is easier and cost efficient as compared to harvesting a single microbial cell and protein therefrom as it requires highly efficient micro-scale laboratory equipment. In other words, obtaining cell density sufficient and feasible for production of food ingredient nutritional supplement from microbial cells is important and is achieved by cultivating microbial cells in optimum growth conditions.

Notably, the microbes have ability to grow in different types of growth conditions ranging from aerobic, anaerobic, and facultative conditions. Moreover, the microbes grow in their suitable natural environments or artificial systems. The artificial system is configured to mimic the natural environment suitable for a given microbes. Typically, an inoculum (i.e. a small amount) of microbes that works as starting material is used for growing more microbes under optimal growth conditions in the artificial system. Further, the microbes are allowed to grow, in a controlled environment, for a defined period of time to achieve an optimum growth (namely, biomass). The optimal growth of the microbes pertains to its biomass or by-product of the microbial growth, to be subsequently harvested for later use, such as for example in human nutrition including proteins, lipids, carbohydrates, vitamins, minerals, fibre, and so forth, and in the present case for producing nutritional supplement from microbial cells.

Optionally, the artificial system is implemented as a bioreactor for example. The term “bioreactor” refers to a vessel intended for biological and/or biochemical reactions required for culturing an inoculum of microbial cells (from a stock solution, for example), growing microbes, and production of biomolecules serving to meet nutritional, pharmaceutical or energy needs of consumers, under defined and controlled physical and chemical conditions. The controlled conditions necessary for obtaining the biomass include optimum gases (such as carbon dioxide, hydrogen and oxygen), minerals, and a liquid phase. Optionally, in the bioreactor, the microbial cell growth is continuous, and the biomass is extracted out of the bioreactor, continuously or batch-wise, when target cell density of biomass is achieved, such as for example for production of nutritional supplement from microbial cells. In an exemplary implementation, biomass cultivation process is performed in the bioreactor with microbial cells loaded into the bioreactor from a stock solution. The biomass is cultivated in a growth medium comprising a carbon source, a nitrogen source, an energy source, minerals and other specific nutrients depending on the microbe, and under controlled conditions. In the bioreactor, the cell growth is continuous, and the biomass leaves the bioreactor when target cell density is reached. The harvested biomass is subjected to processing steps to reduce or remove the microbial endotoxins from the biomass or a final product obtained from the processed biomass. It will be appreciated that the final product may, for some applications, be dehydrated only partially or not at all.

The biomass comprises a gram-negative bacteria. The Gram-negative bacteria are bacteria that do not retain crystal violet stain used in the gram-staining method. Gram-negative bacteria form a part of our environment are ingested or inhaled daily. The Gram-negative bacteria include Escherichia coli, Pseudomonas aeruginosa, Chlamydia trachomatis, Yersinia pestis, and so on. The Gram-negative bacteria comprises an outer membrane that protects the bacteria from antibiotics, detergents, lysozyme, and other physical or chemical degradation means. Additionally, the outer membrane comprises a complex lipopolysaccharide (LPS).

LPS are complex molecules with a lipid region covalently linked to a polysaccharide region. It forms a dense, slightly negatively charged network on the outer membrane of the cell wall shielding the cell from compounds and other cells that may damage the cell inside, yet the layer is loose enough to let nutrients in (as described by Wassenaar and Zimmerman, 2018). From a structural point of view, three different regions are distinguished in the LPS. First, the lipid A, it is the only lipidic part of the molecule and its fatty acids allow the molecule to be inserted in the outer membrane of the microbial cell, like an anchor. Second, the core, it is an oligosaccharide that is linked to the lipid moiety and contains rare and specific carbohydrates. Third, the O-chain that consists of a repeating unit of oligosaccharides making a long carbohydrate chain that can be therefore considered as the O-antigenic polysaccharide. Notably, the toxicity of LPS is mainly due to lipid A, while the polysaccharides are less toxic. Some of the LPS in the microbial cells are endotoxins, however, not all LPS carry toxic activities and therefore cannot be considered as endotoxins. Notably, some fatty acids of the lipid A are β hydroxylated and are considered as specific markers of endotoxins because they have not been described in other biological molecules.

Endotoxins are LPS (made up of a lipid region and a polysaccharide region) in the outer membranes of the Gram-negative bacteria. Human pathogens (Salmonella, Neisseria, Yersinia, Bordetella, and so forth), commensal bacteria (Escherichia, Bacteroides, Veillonella, and so forth) or even environmental bacteria (Rhizobia, Xanthomonas, some Pseudomonas, and so forth) contain the endotoxins. Endotoxins are potent stimulators of the immune system if released in sufficiently large quantities in blood. Specifically, the lipid moiety (namely, Lipid A) of the endotoxins contributes to the toxic activity of the endotoxins. It will be appreciated that while all endotoxins are LPS, all LPS are not endotoxins. Therefore, it is important to prevent the microbial endotoxins from entering the body of the humans or animals. In this regard, the biomass is processed to reduce the endotoxins therefrom to provide a safer and suitable final product (namely, nutritional supplement from microbial cells) to the consumers. It will be appreciated that generally the Gram-positive bacteria do not need endotoxin removal. However, normally, when cultivating the Gram-positive bacteria, the Gram-negative bacteria could also be present when pure cultures are not necessary. In such case, the culture of said Gram-positive bacteria is required to processed to reduce the microbial endotoxins derived from the Gram-negative bacteria.

Optionally, the microbial cells comprise isolated bacterial strain deposited as VTT-E-193585 or a derivative thereof. The said isolated bacterial strain or a derivative thereof is a Gram-negative bacterium that is inherently a source of microbial endotoxins. It will be appreciated that the said isolated bacterial strain or a derivative thereof is genetically stable and can be grown in a broad range of process conditions, ranging from optimal to stressful conditions, over time. The term “genetically stable” as used herein, refers to a characteristic of a species or a strain/isolate to resist changes and maintain its genotype over multiple generations or cell divisions, ideally hundreds to thousands. Optionally, the said isolated bacterial strain or a derivative thereof utilize hydrogen gas as energy source and carbon dioxide as carbon source. Beneficially, the said strain or the derivative thereof comprises iron and vitamin B12. Moreover, the final product resulting from the said strain or the derivative thereof does not have a bean-off-flavor and is therefore easier to flavor. Possibly, the final product also has umami (namely, savory or “meat-like”) flavor. Furthermore, the final product yields a nutritional supplement high in protein upon further downstream processing thereof.

The term “downstream processing” as used herein refers to the process that follows the microbial cell cultivation in the bioreactor for obtaining the biomass. The downstream processing of the biomass enables reducing or removing microbial endotoxins by subjecting the biomass to conditions, physiological and mechanical, to provide a final product comprising reduced or no endotoxins and is safe for use by the consumers, humans and animals alike. Typically, the downstream processing comprises steps of heat treatment (referred to as “incubating” hereafter), hydrolysis, concentrating (referred to as “separating” hereafter), optionally homogenizing and dehydrating (referred to as “drying” hereafter), and so forth. It will be appreciated that the downstream process is a step of the method for producing nutritional supplement from microbial cells as disclosed in the present disclosure.

In this regard, the method comprises, after obtaining biomass, incubating the biomass with a heat treatment at temperature from 55° C. up to 80° C. for an incubation time from 10 minutes up to 60 minutes. The term “incubating” or “incubation” as used herein refers to heat treatment performed at a selected temperature for a selected incubation time. In other words, incubating subjects the biomass to suitable conditions such as temperature, and optionally other complementing conditions including humidity and atmospheric composition for example, for allowing growth of microbes and further development of the biomass. Notably, incubating the biomass enables the microbes to grow through different phases in their lifecycle, such as for example lag phase, log phase, stationary phase and decline phase. It will be appreciated that the biomass in its late log phase or early stationary phase is typically used for its nutritional benefits such as for example producing nutritional supplement from microbial cells as disclosed in the present disclosure. Beneficially, incubating at suitable temperatures enable the microbes to undergo certain chemical and structural changes to enable their further downstream processing. Incubating the biomass with heat treatment at temperature from 55° C. up to 80° C. for an incubation time from 10 minutes up to 60 minutes results in hydrolysis of the outer membrane of the cell wall structures of the microbes leading to partial removal of the LPS-containing components (such as endotoxins) during further processing of the biomass, such as during a separation stage thereof. The incubation temperature may for example be from 55, 56, 57, 58, 59, 60, 65, 70 or 75° C. up to 56, 57, 58, 59, 60, 65, 70, 75 or 80° C. The incubation period may for example be from 10, 15, 20, 25, 30, 35 or 40 minutes up to 20, 25, 30, 35, 40, 55 or 60 minutes. Moreover, outer membrane cell wall partial degradation results in a final product with at least 10-1000 times lower endotoxin response. Furthermore, the said temperature range ceases metabolic activity of the microbe, for example, the said temperature range inactivates the biomass.

Optionally, the incubation temperature is from 55° C. up to 70° C., or preferably from 60° C. up to 68° C. The incubation temperature may for example be from 55, 56, 57, 58, 59, 60, 61, 62, 63, 64, 65, 66 or 67° C. up to 61, 62, 63, 64, 65, 66, 67, 68, 69 or 70° C. The incubation temperature may for example be from 60, 61, 62, 63, 64, 65, 66 or 67° C. up to 61, 62, 63, 64, 65, 66, 67 or 68° C.

Optionally, the incubation time is from 15 minutes up to 40 minutes, or preferably from 20 minutes up to 30 minutes. The incubation period may for example be from 15, 16, 17, 18, 19, 20, 22, 24, 26, 28, 30 or 35 minutes up to 20, 22, 24, 26, 28, 30, 35 or 40 minutes. The incubation period may for example be from 20, 22, 24, 26 or 28 minutes up to 22, 24, 26, 28 or 30 minutes.

Beneficially, the aforesaid incubation of the biomass with selected temperature ranges and incubation time results outer membrane of cell wall structure hydrolysis leading to partial removal of LPS from the cell wall. Moreover, incubation of the biomass at temperature from 60° C. up to 68° C. and incubation time from 20 minutes up to 30 minutes resulted in most efficient removal of LPS from the cell wall. Optionally, incubating the biomass is selected to be at least one of a batch incubation, a continuous incubation. Notably, the batch incubation relates to incubating the biomass in batches for a longer predetermined incubation time, such that the total amount of biomass incubated is withdrawn together, i.e. all-in all-out, and then starting afresh for the next incubation cycle. The continuous incubation relates to shorter incubation times and routinely withdrawing the biomass for further processing and obtaining the end product, namely nutritional supplement therefrom.

Moreover, the method comprises concentrating the biomass by separating and removing a liquid phase from a solid phase to obtain a dry matter content from 2% up to 40% of total weight of the nutritional supplement. The dry matter content of total weight of the nutritional supplement may be for example from 2, 4, 6, 8, 10, 15, 20, 25, 30 or 35% up to 6, 8, 10, 15, 20, 25, 30, 35% up to 40%. The higher content of the dry matter leads to higher level of proteins in the nutritional supplement. However, if the dry matter content of the total weight of the nutritional supplement is more than 40%, the structure of the nutritional supplement in not easily flowing and therefore difficult to use in methods of producing food products. Concentrating the biomass comprising gram-negative bacteria enables removing excess liquid phase from solid phase of the biomass. The liquid phase of the biomass comprises the hydrolysed components of the cell wall structures including the LPS-containing endotoxins. Separating and removing liquid phase from the solid phase leaves the concentrated biomass with reduced endotoxins therein. During the incubation, some of the LPS is released and leaks out from the cells. During separation, most of the liquid phase is removed, which leads to removing LPS present in the liquid phase.

Optionally, the dry matter content is from 5% up to 30%, or preferably from 8% up to 25% of total weight of the nutritional supplement. The dry matter content of total weight of the nutritional supplement may be for example from 5, 6, 7, 8, 9, 10, 15 or 20% up to 8, 10, 15, 20% or 25%.

Optionally, the separating is carried out with a separation method selected from at least one of a centrifugation, a filtration. Centrifugation typically is a technique for the separation of particles from a solution, comprising a solid phase and a liquid phase, according to their size, shape, density, viscosity or speed of rotor employed for separation. In this regard, the solution is placed in a centrifuge tube that is then placed in rotor and spun at a definite speed. Optionally, centrifugation is performed with a centrifugal force ranging between 10000×g and 20000×g. The centrifugation separates about 90-95% of liquid phase from the solid phase of cultivation broth. It will be appreciated that centrifugation is the most efficient and easiest way to separate the liquid and solid phases, thereby removing endotoxins that mostly present in liquid phase from the concentrated biomass. The filtration technique typically separates the liquid and solid phases through a semi-permeable membrane that allows the liquid phase to pass therethrough while retaining the solid phase over the said semi-permeable membrane. Optionally, the filtration can be done with a ceramic filter. The filtration provides the most energy-efficient way to separate the liquid and solid phases, thereby removing endotoxins that mostly present in liquid phase from the concentrated biomass.

The method comprises homogenizing the nutritional supplement with high-pressure homogenization at least one run for at least partially degrading walls of the microbial cells. The term “homogenizing” as used herein refers to a means of physical disruption of microbial cell walls. It will be appreciated that incubating the microbial cells partially disrupts the outermost membrane of microbial cell walls, and homogenizing the biomass further disrupts the microbial cell walls. High-pressure homogenization (HPH) is the most effective technique for microbial cell wall disruption by physical (mechanical) disruption. It will be appreciated that cell wall disruption resulting from homogenizing the biomass degrades partially the remaining endotoxins from the microbial cells. The high-pressure homogenization process results in partial lysis of cell and increasing soluble protein content of the biomass thereby improving functional properties of the biomass as a food ingredient. Additionally, homogenizing the biomass further removes endotoxins remaining post the separation process thereby further reducing from the biomass. Therefore, homogenizing reduces endotoxin levels in the nutritional supplement produced from microbial cells and containing gram-negative bacteria, making the nutritional supplement safe for human consumption.

Typically, homogenizing techniques exploit fluid flow, particle-particle interaction, and pressure drop to facilitate cell disruption. The term “high-pressure homogenization” as used herein refers to a physical or mechanical process of forcing a stream of sample, such as the biomass that comprises solid-phase and liquid phase (remining post separation process), through a system, implemented as a homogenizing device (discussed in details later) that subjects the sample to a plurality of forces, such as high pressure or any combination of shear forces for example, which intend to homogenize the sample and/or reduce the particle size of any components within the sample

Optionally, the homogenizing is carried out as high-pressure homogenization for at least one run at pressure from 800 bars up to 2000 bars. Optionally, homogenization pressure may typically be from 800, 1000, 1200, 1400, 1600 or 1800 bars up to 1000, 1200, 1400, 1600, 1800 or 2000 bars. The term “at least one run” as used herein refers to the number of cycles or passes the concentrated biomass is subjected to increase cell disruption efficiency. Optionally, the concentrated biomass is subjected to the aforementioned homogenization pressure of 800-2000 bars once, twice or thrice. It will be appreciated that during the homogenization process, proteins are partially released from the microbial cell and into the remaining liquid phase of the biomass (i.e. after the separation process). Beneficially, the homogenization process results in partial lysis of cell and increasing soluble protein content of the biomass thereby improving functional properties of the biomass as a food ingredient. Additionally, homogenizing the biomass further removes endotoxins remaining post the separation process thereby further reducing from the biomass.

Optionally, the high-pressure homogenization is carried out at pressure from 900 bars up to 1000 bars. Optionally, homogenization pressure may typically be from 900, 910, 920, 930, 940, 950, 960, 970, 980 or 990 bars up to 910, 920, 930, 940, 950, 960, 970, 980, 990 or 1000 bars. The said range of homogenization pressure provides best results with increased soluble protein content and decreased endotoxin levels in the homogenized biomass and the ratio of soluble protein content compared to decreased endotoxins levels is optimal.

Optionally, the method comprises drying the nutritional supplement to obtain dry matter content from 94% up to 99% of the total weight of the nutritional supplement. The term “drying” as used herein refers to a process of drying out liquids from raw materials, such as biomass. Optionally, drying of biomass is accomplished by subjecting the biomass to either relatively low temperatures over rotating the biomass in a closed system, such as a drying drum for example, or rapidly drying using a hot gas. It will be appreciated that drying biomass enables easy and effective storage of the nutritional supplement or the powder from derived therefrom. Moreover, drying the biomass prevent the nutritional supplement or the powder from a potential infestation and thereby unfit for consumption by humans or animals. Furthermore, drying the biomass increases the dry matter content of the nutritional supplement. The dry matter content of the nutritional supplement post drying process is typically from 94, 94.5, 95, 96, 97 or 98% up to 94.5, 95, 96, 97, 98 or 99%. Optionally, the drying process is followed by homogenizing of the final product to obtain a powder form of the final product, i.e. the nutritional supplement.

Optionally, the drying is selected as at least one of a drum drying or a spray drying and drying temperature is from 120° C. up to 180° C. The drum drying is a process of rotating the nutritional supplement in rotating, high-capacity drying drums, at relatively low temperatures to produce sheets of drum-dried product. The sheets of drum-dried product are subsequently milled to a finished final product, i.e. the nutritional supplement. Beneficially, the drum drying is suitable for highly viscous samples which cannot be dried using other drying techniques. The spray drying utilizes a spray of hot gases (such as nitrogen or oxygen) to rapidly dry the biomass. Spray dying is suitable for drying thermally-sensitive samples, such as food and pharmaceutical products. The dried nutritional supplement contains reduced levels of endotoxins after the incubation, separation and homogenization steps as compared to the samples that are not incubated, and/or not homogenized. Moreover, the nutritional supplement has endotoxin levels reduced to food-acceptable levels. The drying is typically carried out at drum temperatures ranging from 120, 125, 130, 135, 140, 150, 160 or 170° C. up to 125, 130, 135, 140, 150, 160, 170 or 180° C. It will be appreciated that drying at the aforesaid temperature range dries out liquid (or water) in the nutritional supplement to obtain powder form thereof. The powder form is easy to store and has a longer shelf-life.

Moreover, the aforesaid temperature range kills unwanted microbes or pathogens that could grow along with the nutritional supplement during any of the aforementioned cultivation and/or steps of downstream processing of biomass. Furthermore, drying the nutritional supplement facilitates efficient grinding thereof to obtain the final product with a desired particle size.

Optionally, the method further comprises extruding the nutritional supplement with high-moisture extrusion. The term “extruding” as used herein refers to a process of shaping a material, such as a food product, into a product of a fixed cross-section (desirable form), such as slices, blocks, pieces, cubes, and so forth. In this regard, the material is forced through a die of the desired cross-section and subjected to compressive and shear stresses. The term “high-moisture extrusion” as used herein refers to a thermo-mechanical cooking process often used for production of high moisture meat analogues (HMMA). Typically, HMMA are made from plant-based ingredients to imitate meat-like texture and mouthfeel. The HMMA products may show fibrillar structure resembling meat fibres, for example. The relevant products are texturized vegetable proteins (TVP) used in vegan nuggets and burgers, or seitan-type products. The HMMA product may further be mixed with other ingredients, such as spices, nutrients, pharmaceuticals and the like, to enhance the nutritional and flavours of the HMMA product.

Moreover, HMMA product produced from dried nutritional supplement using high-moisture extrusion showed dramatically lowered endotoxin levels compared to the level thereof in the nutritional supplement itself. High-moisture extrusion can reduce the endotoxins from >4000 EU/g to <0.5 EU/g in the HMMA products. Moreover, high-moisture extrusion of the nutritional supplement resulted in a HMMA product which showed no endotoxic response.

In some cases, extruding the biomass may be followed by storing the nutritional supplement in containers, such as cans or pouch packets, which evacuated and sealed at its end. Beneficially, proper storing prevents post-processing contamination of the product. Non-hygienic processing or post-processing contamination is a cause for increased endotoxin levels in fresh and other food produce in general.

The present disclosure also relates to the system as described above. Various embodiments and variants disclosed above apply mutatis mutandis to the system.

Optionally, the bioreactor may have a shape such as cylindrical, conical, cuboidal or cubical. Optionally, the volume of the bioreactor is for example 10 litres, 100 litres, 200 litres, 1000 litres, and so forth. Optionally, the bioreactor is fabricated from a material that is inert to contents to be processed in the bioreactor. In an example, the fabrication material may be stainless steel (for example type 304, 316 or 316 litres), other suitable metals or alloys, glass material, fibres, ceramic, plastic materials and/or combinations thereof. Moreover, the fabrication material is typically waterproof and strong enough to withstand abrasive effects of various biological, biochemical and/or mechanical processes, such as micro-organisms concentrations, biomass productions, agitation forces, aeration forces, operating pressures, temperatures, acids, alkali and so forth. Typically, the bioreactor has an adequate thickness to hold a weight of the contents therein, and carry out various biological, biochemical and/or mechanical processes. Furthermore, the bioreactor should preferably be such that it withholds the sterilisation conditions.

Incubation of the biomass is performed in a typical heat-exchanger. The heat-exchanger is a system user to transfer heat between two or more fluids. In this regard, the heat exchanger may have flow arrangement selected from either parallel flow or counter-flow, such that the heat-exchanging fluids travel in parallel to one another or the heat-exchanging fluids travel in directions opposite to one another. Usually, heat-exchanger are widely used in industrial scales and are well known to a person skilled in the art.

Optionally, the heat-exchanger is selected to be at least one of a tank heat-exchanger, a tubular heat-exchanger, or a plate heat-exchanger. Optionally, the tank heat-exchanger is for example a jacketed tank heat-exchanger. The jacketed tank heat-exchanger is a heating vessel designed for heating (or cooling) the content therein by using a heating (or cooling) “jacket” around the heating vessel through which a heating (or cooling) fluid is circulated. The tubular heat-exchanger comprises a set of tubes containing a fluid to be heated (or cooled) and a second fluid (for heat exchanging) runs over the set of tubes to provide (or absorb) the heat. The tubular heat-exchangers are suitable for high-pressure applications. The plate heat-exchanger contains a plurality of heat transfer plates bundled together in a gasket arrangement, wherein each pair of plates provides two separate channel system for the fluid to flow through.

Separation of liquid and solid phases of the biomass can be performed in centrifugal separators or filtration units, for example membrane filters or ceramic filters, widely used in industrial scales. The centrifugal separator typically uses centrifugation technique for separating components in a solution based on a particle size thereof. The membrane filtration units operate based on filtration techniques that utilize a semi-permeable membrane to separate solid and liquid phases of a solution. It will be appreciated that centrifugal separators are preferred for concentrating bacterial cells as compared to filtration.

The homogenizing device employs physical or mechanical ways of disrupting or homogenizing materials. Typically, used homogenizing devices include mortar and pestle, blenders, bead mills, sonicators, rotor-stator, and the like. The high-pressure homogenization (HPH) includes at least one pass, for example 1, 2, or 3, through the homogenizing device to increase cell disruption efficiency.

Optionally, the homogenizing device is a high-pressure homogenizer and wherein the pressure in the high-pressure homogenizer is from 800 bars up to 2000 bars or preferably from 900 bars up to 1000 bars. The high-pressure homogenizers typically use very high pressures to disrupt the cell structures. Optionally, the homogenizing device is a liquid mill. The liquid mill typically uses shear forces to disrupt the cell structures.

The biomass is dried in dryer. Typically, the dryers employ direct or indirect heat supply to dry out the liquid phase of the sample, herein the nutritional supplement. In this regard, the dyer uses a stream of gases heated (or cooled such as in freeze-drying) using various techniques known to a person skilled in the art. Optionally, the dryer is selected to be at least one of a drum dryer or a spray dryer. The drum dryer is a rotating, high-capacity vessel configured to contain the slurry material, such as the biomass, and rotate the material therein at relatively low temperatures to produce sheets of drum-dried product. The drum-dried product may be further milled or finished to a flake or powder form. Optionally, the drum dyer is a double drum dryer that provides a steam pressure in a range of 2 to 7 bars. The pressure in a double drum dryer may for example be from 2, 2.5, 3, 3.5, 4, 4.5, 5, 5.5 or 6 bars up to 2.5, 3, 3.5, 4, 4.5, 5, 5.5, 6, 6.5 or 7 bars. The spray dryers enable rapidly drying the slurry material by using a hot gas. Spray drying is typically suitable for thermally-sensitive materials.

Optionally, the system further comprises an extruder for extruding the nutritional supplement with high-moisture extrusion. The extruder is typically a system having a defined (or fixed) cross-section that is used to pass a material therethrough to provide a shape or the desired cross-section to the material. The extruder usually uses friction (between the passing material and the extruder) and heat to due to pressure generated as a result of friction to shape the final product being extruded from the extruder.

Optionally, the microbial cells comprise isolated bacterial strain deposited as VTT-E-193585 or a derivative thereof.

### EXPERIMENTAL RESULTS

Limulus Amoebocyte Lysate (LAL) assay: The LAL assay is a calorimetric method for identifying and quantifying the microbial endotoxins in a sample. LAL assay is very sensitive and is required by the Pharmacopeia for products release in the market. The LAL assay operates by an enzymatic cascade from amoebocyte cells of the Horseshoe crab that is able to coagulate the lymph in presence of endotoxins. Based on a universal Reference Standard Endotoxin (RSE), prepared from an Escherichia coli strain, the following value has been established: 0.2 ng of LPS=1 Endotoxin Unit [EU]. In this regard, it will be appreciated that the LAL assay only aims at measuring an activity and its conversion to quantity is only based on the E. coli strain LPS response. Also, all LPS being structurally different react differently to the LAL assay. Moreover, as the LAL assay is a colorimetric method, therefore it cannot be used if the material is not soluble. For assaying an insoluble medical device a protocol defined by the European Pharmacopeia is required to be followed. Based on the parameters defined in the said regulation, powder samples were subjected to extraction prior to the LAL assay as by dispersing in water, agitation for a defined amount of time, centrifugation, filtration of the supernatant. The filtrate was subjected to the LAL assay for quantification of the endotoxins therein.

The LAL assay was used by the Applicants of the present disclosure for the analysis of the microbial biomass sample obtained from cultivation of microbial cells and the nutritional supplement samples obtained through different downstream processing routes of the biomass. All nutritional supplements, irrespective of the downstream processing route indicated toxic activity (measured as Endotoxin Unit per gram of sample (EU/g)) higher than milk powder for example, but comparable to lactic acid bacteria tablets or Spirulina powder analyzed within the same sets. Table 1 below provides results of LAL assay performed on several microbial biomass samples showing high endotoxic activity (100000-3000000 EU/g) and its reduction via downstream processing thereof. The LAL assay values represent repeats of same samples using Kinetic turbiditimetric or chromogenic LAL assays. Day 1 and 2 nutritional supplement samples are the spaced 12 days apart and the Day 2 and 3 nutritional supplement samples are spaced 30 days apart. As shown, the nutritional supplement samples which did not go through incubation and high-pressure homogenization (HPH) before drying (i.e. sample V0) showed intermediate endotoxic activity (i.e. 6000-15000 EU/g).

However, the nutritional supplement sample which were subjected to incubation and HPH process (i.e. sample V1) before drying showed clear decrease in endotoxic activity (i.e. to values <50-200 EU/g).

TABLE 1
LAL assay performed on several microbial biomass samples

| Nutritional | Sample info | LAL assay | LAL assay | LAL assay |
|---|---|---|---|---|
| supplement | (Temperature, | result | result | result |
| Sample | time) | (EU/g) | (EU/g) | (EU/g) |
| V0; Day 1 | No incubation, No | — | 180000 | — |
| | High-pressure | | | |
| | Homogenization | | | |
| V1; Day 1 | Incubated at 60° C. | — | 1400 | — |
| | for 32 min, and 3 | | | |
| | cycles of High- | | | |
| | pressure | | | |
| | Homogenization | | | |
| | at 900 bars | | | |
| V0; Day 2 | No incubation, No | 410000 | 2860000 | 72000 |
| | High-pressure | | | |
| | Homogenization | | | |
| V0.5; Day 2 | No incubation, 2 | 65000 | 170000 | 12000 |
| | cycles of High- | | | |
| | pressure | | | |
| | Homogenization | | | |
| | at 900 bars | | | |
| V1; Day 2 | Incubated at 68° C. | 500 | 60000 | 6000 |
| | for 22 min, and 2 | | | |
| | cycles of High- | | | |
| | pressure | | | |
| | Homogenization | | | |
| | at 900 bars | | | |
| V0; Day 3 | No incubation, No | — | 38200 | 4000 |
| | High-pressure | | | |
| | Homogenization | | | |
| V0.5; Day 3 | No incubation, 2 | — | 5110 | <0.5 |
| | cycles of High- | | | |
| | pressure | | | |
| | Homogenization | | | |
| | at 900 bars | | | |
| Lactic acid | | 91000 | 1000 | — |
| bacteria | | | | |
| Spirulina | | 98000 | 121000 | 83000 |
| Milk powder | | — | 540 | <0.5 |
| Probiotic | | — | — | out of |
| capsules of | | | | range |
| E. coli Nissle | | | | |

The LAL assay was also performed on the separator supernatants (referred to as the separated liquid phase above). It was observed that the separated liquid phase of the incubated biomasses (V1) repeatedly showed higher EU/mL values as compared to that of the non-incubated batches (V0). Thereby, indicating that the LPS is detached from the outer cell membrane during incubation and removed with the separated liquid phase during the separation step.

Table 2 below provides LAL assay results of two nutritional supplement samples and their respective extrudates (obtained after high-moisture extrusion).

TABLE 2
LAL assay results of nutritional supplement
samples and their respective extrudates

| | Sample info | LAL test result |
|---|---|---|
| Sample | (Temperature, time) | (EU/g) |
| V0; Day 2 | No incubation, No HPH | 72000 |
| V0.5; Day 2 | No incubation, + HPH 2 runs | 12000 |
| Extrudate | Extrudate of V0 | <0.5 |
| Extrudate | Extrudate of V0.5 | 2 |
| Spirulina | | 83000 |
| Milk powder | | <0.5 |

### DETAILED DESCRIPTION OF THE DRAWINGS

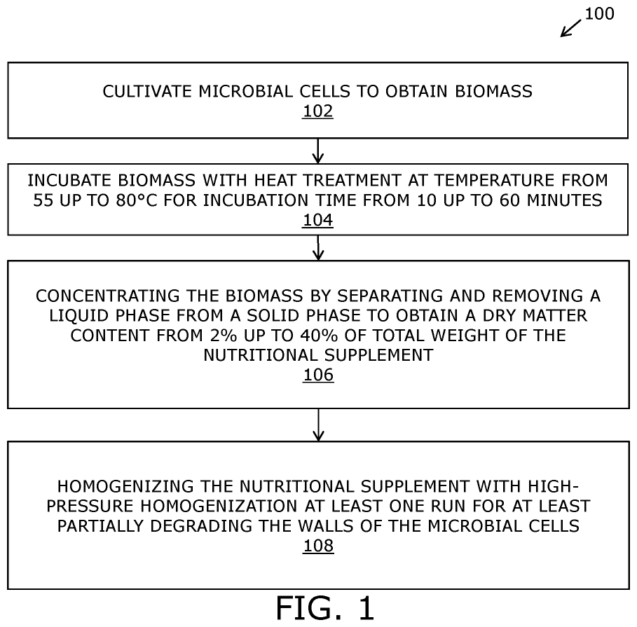

Referring to FIG. 1, there is shown a flowchart 100 illustrating steps of a method for producing nutritional supplement from microbial cells, in accordance with an embodiment of the present disclosure. At step 102, microbial cells is cultivated to obtain a biomass. At step 104, the biomass is incubated with a heat treatment at temperature from 55° C. up to 80° C. for an incubation time from 10 minutes up to 60 minutes. At step 106, the biomass is concentrated by separating and removing a liquid phase from a solid phase to obtain a dry matter content from 2% up to 40% of total weight of the nutritional supplement. At step 108, the nutritional supplement is homogenized with high-pressure homogenization at least one run for at least partially degrading the walls of the microbial cells.

The steps 102, 104, 106 and 108 are only illustrative and other alternatives can also be provided where one or more steps are added, one or more steps are removed, or one or more steps are provided in a different sequence without departing from the scope of the claims herein.

It may be understood by a person skilled in the art that the FIG. 1 is merely an example for sake of clarity, which should not unduly limit the scope of the claims herein. The person skilled in the art will recognize many variations, alternatives, and modifications of embodiments of the present disclosure. In an example, the processes described in steps 102, 104 and 106 may follow a different sequence to reach the final product.

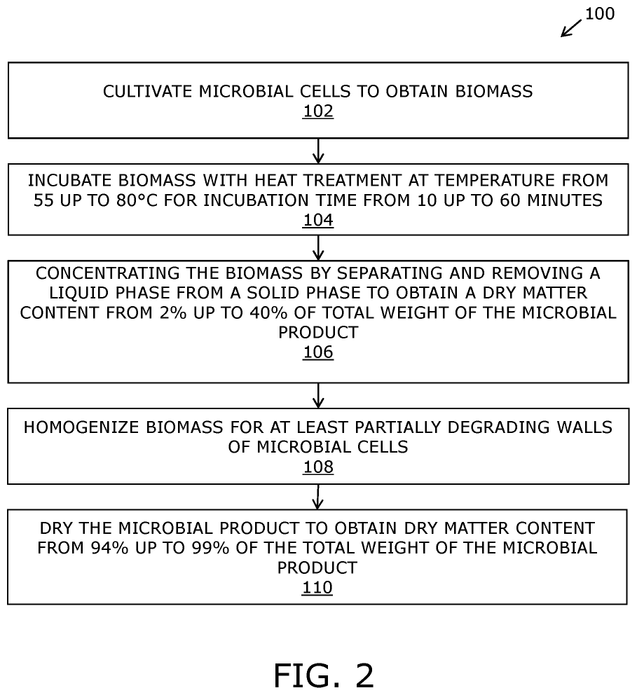

Referring to FIG. 2, there is shown a flowchart 100 illustrating steps of a method for producing protein powder, in accordance with an optional embodiment of the present disclosure. At step 102, microbial cells are cultivated to obtain a biomass. At step 104, the biomass is incubated with a heat treatment at temperature from 55° C. up to 80° C. for an incubation time from 10 minutes up to 60 minutes. At step 106, the biomass is concentrated by separating and removing a liquid phase from a solid phase to obtain a dry matter content from 2% up to 40% of total weight of the nutritional supplement from microbial clls. At step 108, the biomass is homogenized for at least partially degrading walls of the microbial cells. At step 110, the biomass is dried to obtain dry matter content from 94% up to 99% of the total weight of the nutritional supplement.

The steps 102, 104, 106, 108 and 110 are only illustrative and other alternatives can also be provided where one or more steps are added, one or more steps are removed, or one or more steps are provided in a different sequence without departing from the scope of the claims herein.

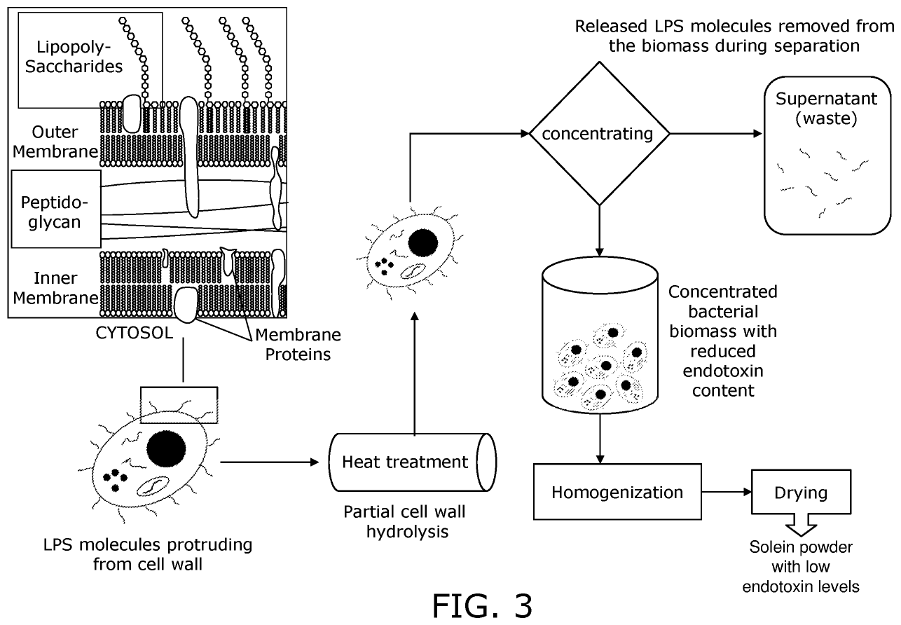

Referring to FIG. 3, there is shown an illustration of low-endotoxin bacterial biomass production process, in accordance with an embodiment of the present disclosure. As shown, the microbial cell well comprises the lipopolysaccharide-containing endotoxins represented as protruding lines from the cell wall of the microbial cell. The microbial cells are cultivated for example in a bioreactor (not shown). The microbial cells (represented as a single microbial cell) are is incubated with heat-treatment to partially hydrolyze the outer membrane of cell wall thereof. The partially hydrolyzed microbial cell is subsequently run through a separator to separate the liquid phase of the microbial cell containing the endotoxins removed from the cell wall during partial hydrolysis thereof. The solid phase of the microbial cell is concentrated with reduced endotoxin content due to separation of the liquid phase containing the partially removed endotoxins. Subsequently, the concentrated microbial cells (or the biomass) is subjected to homogenization and later to drying. Upon drying, biomass is obtained in powder form with low endotoxin levels, usually 10-1000 times lower endotoxin response as measured using assay methods, such as the LAL assay as described above.

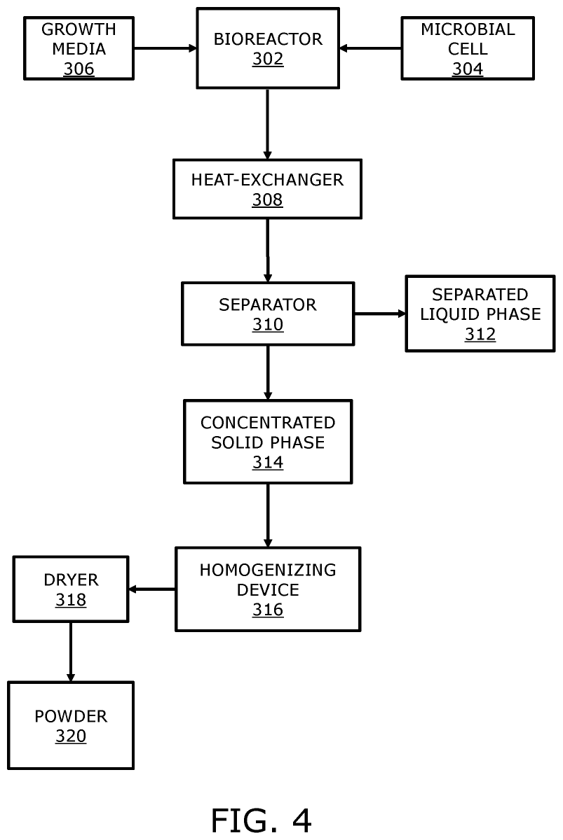

Referring to FIG. 4, there is shown a block diagram of a system 300 for producing nutritional supplement from microbial cells 320, in accordance with an embodiment of the present disclosure. The system 300 comprises a bioreactor 302 for cultivating microbial cells 304 to obtain a biomass, wherein the bioreactor is provided with a growth media 306 and microbial cells 304 for growth thereof; a heat-exchanger 308 for incubation at temperature from 55° C. up to 75° C. for an incubation time from 15 minutes up to 40 minutes; a separator 310 for separating a liquid phase 312 from a solid phase 314 of the biomass; a homogenizing device 316 for degrading at least partially walls of the microbial cells 304 of the biomass; and a dryer 318 for drying the biomass to obtain a powder 320 form of the biomass having a dry matter content from 94% up to 99%.

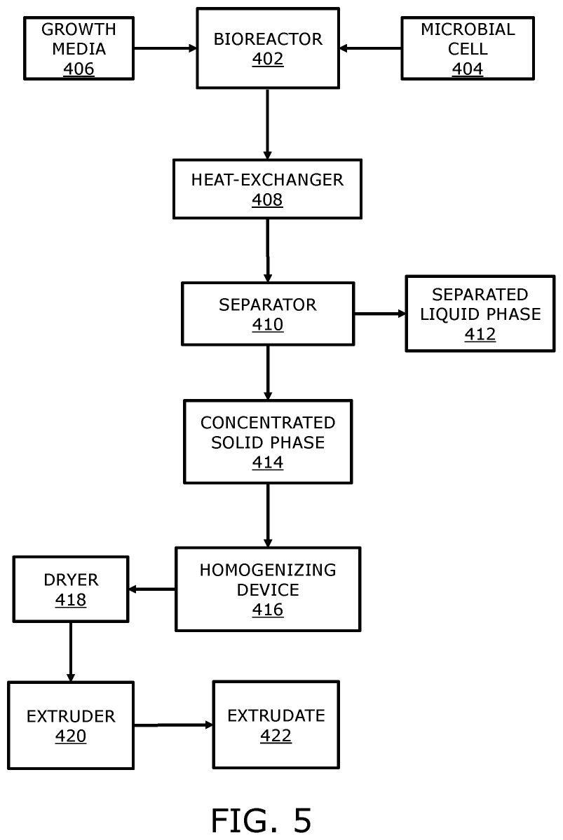

Referring to FIG. 5, there is shown a block diagram of a system 400 for producing a high-moisture meat analogue 422, in accordance with an embodiment of the present disclosure. The system 400 comprises a bioreactor 402 for cultivating microbial cells 404 to obtain a biomass, wherein the bioreactor is provided with a growth media 406 and microbial cells 404 for growth thereof; a heat-exchanger 408 for incubation at temperature from 55° C. up to 75° C. for an incubation time from 15 minutes up to 40 minutes; a separator 410 for separating a liquid phase 412 from a solid phase 414 of the biomass; a homogenizing device 416 for degrading at least partially walls of the microbial cells 404 of the biomass; a dryer 418 for drying the biomass; and an extruder 420 for extruding the biomass with high-moisture extrusion after drying the biomass to yield the high-moisture meat analogue 422.

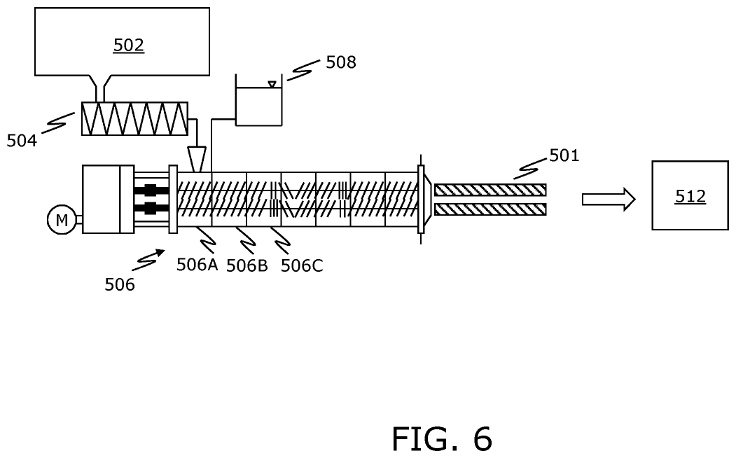

Referring to FIG. 6, there is shown a schematic illustration of a process of producing a high-moisture meat analogue 510, in accordance with an embodiment of the present disclosure. As shown, the biomass sample 502 is dried in a dryer 504 and passed through an extruder 506. The extruder 506 is fed with water from a water feed 508 to provide moisture to the dried biomass sample. The extruder 506 is configured to extrude the biomass sample through a plurality of barrel sections, such as sections 506A, 506B, 506C, in the extruder 506. The extruder 506 comprises, at an end thereof, a cooling die 510 that is configured to operate at a predefined temperature, such as 50° C., and cool the high-moisture meat analogue 512 exiting from the extruder 506 and subsequently the cooling die 510. The high-moisture meat analogue 512 contains no endotoxins. Moreover, the extruder 506 is operated by a motor M.

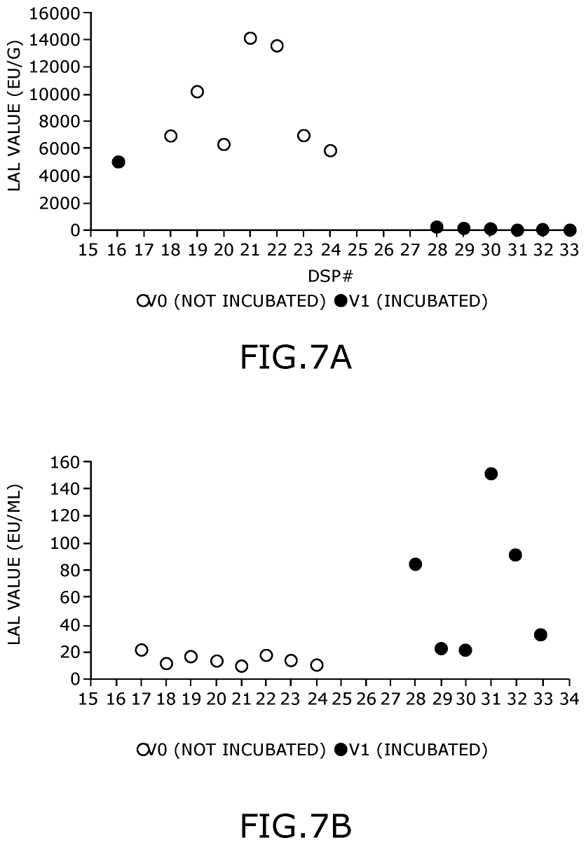

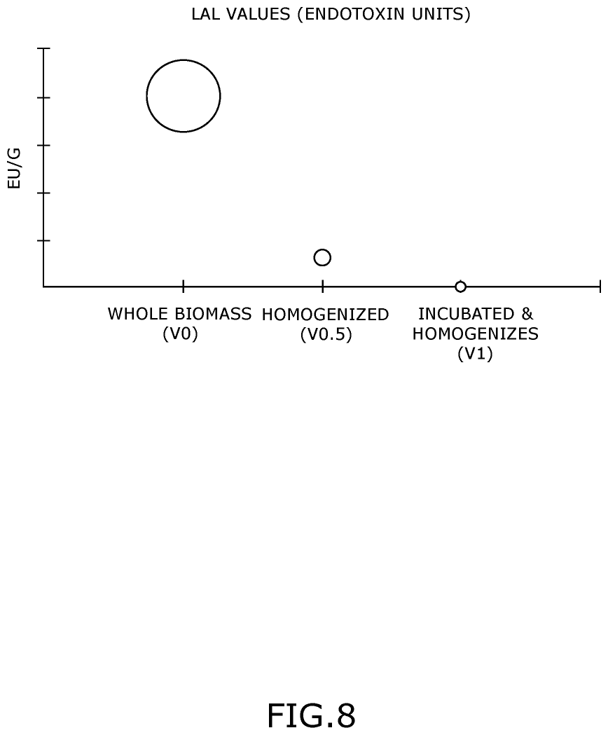

Referring to FIGS. 7A, 7B and 8, shown are the LAL assay results of different samples showing intermediate endotoxic activity in accordance with an embodiment of the present disclosure. FIG. 6A depicts LAL assay results performed on the powder samples. FIG. 6B depicts LAL assay results performed on the separator supernatants or liquid phase containing endotoxin. The LAL assay comprises powder samples to be dispersed in water, vortexed, centrifuged, and the supernatant portion is filtered and mixed with LAL reagent using the kinetic turbidimetric method.

As shown in FIG. 7A, the endotoxic activity is reduced in samples of nutritional supplement samples that have been processed via incubation at a temperature of 68° C. for an incubation time of 30 minutes. As shown, the nutritional supplement samples which did not go through incubation and high-pressure homogenization (HPH) before drying (i.e. sample V0) showed intermediate endotoxic activity, i.e. 6000-15000 EU/g. However, the nutritional supplement samples which were subjected to incubation and HPH process (i.e. sample V1) showed clear decrease in endotoxic activity, i.e. to values <50-200 EU/g.

As shown in FIG. 7B, the endotoxic activity is high in samples of separator supernatant which were subjected to incubation at temperature in a range of 68-75° C. for an incubation time of 20-35 min (i.e. sample V1) as compared to the samples of separator supernatant which were not subjected to incubation (i.e. sample V0).

As shown in FIG. 8, the endotoxic activity as measured from the amount of Endotoxin Units per gram of sample (EU/g) is highest for the whole biomass i.e. not incubated and not homogenized (i.e. V0). Moreover, the EU/g value of the homogenized sample that had not been incubated (i.e. V0.5) is lower as compared to the sample that is not incubated and not homogenized (i.e. V0). Furthermore, the samples which were subjected to incubation and homogenization (i.e. V1) have the least EU/g value. Clear decrease in toxic activity of incubated and homogenized samples (V1) were observed repeatedly as compared to samples which were not gone through incubation and homogenization (V0) or only homogenized (V0.5).

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of producing a nutritional supplement from microbial cells, the method comprising:
cultivating the microbial cells to obtain a biomass comprising gram-negative bacteria;
incubating the biomass for at least partially degrading walls of the microbial cells with a heat treatment at temperature from 55° C. up to 80° C. for an incubation time from 10 minutes up to 60 minutes;
concentrating the incubated biomass by separating and removing a liquid phase from a solid phase to obtain a dry matter content from 2% up to 40% of total weight of the nutritional supplement; and
homogenizing the nutritional supplement with high-pressure homogenization at least one run for at least partially degrading the walls of the microbial cells.

2. A method according to claim 1, wherein the dry matter content is
from 5% up to 30%, or
preferably from 8% up to 25%
of total weight of the nutritional supplement.

3. A method according to claim 1 further comprising drying the nutritional supplement to obtain dry matter content from 94% up to 99% of the total weight of the microbial product.

4. A method according to claim 1, wherein the incubation temperature is
from 55° C. up to 70° C., or
preferably from 60° C. up to 68° C.; and
the incubation time is
from 15 minutes up to 40 minutes, or
preferably from 20 minutes up to 30 minutes.

5. A method according to claim 3 further comprising extruding the nutritional supplement with high-moisture extrusion.

6. A method according to claim 1, wherein the homogenizing is carried out at pressure
from 800 bars up to 2000 bars, or
preferably from 900 bars up to 1000 bars.

7. A method according to claim 1, wherein the separating is carried out with a separation method selected from at least one of a centrifugation, a filtration.

8. A method according to claim 3, wherein the drying is selected as at least one of a drum drying or a spray drying and drying temperature is from 120° C. up to 180° C.

9. A method according to claim 1, wherein the microbial cells comprise isolated bacterial strain deposited as VTT-E-193585 or a derivative thereof.

10. A system for producing a nutritional supplement from microbial cells, the system comprising:
a bioreactor for cultivating the microbial cells to obtain a biomass comprising gram-negative bacteria;
a heat-exchanger having incubation temperature from 55° C. up to 80° C. and incubation time from 10 minutes up to 60 minutes for partially degrading walls of the microbial cells;
a separator for separating a solid phase and a liquid phase to obtain a dry matter content from 2% up to 40% of total weight of the nutritional supplement; and
a high-pressure homogenizer for at least partially degrading the walls of the microbial cells of the nutritional supplement.

11. A system according to claim 10 further comprising a dryer to obtain a dry matter content from 94% up to 99% of the total weight of the nutritional supplement.

12. A system according to claim 11 further comprising an extruder for extruding the microbial product with high-moisture extrusion.

13. A system according to claim 10, wherein the heat-exchanger is selected to be at least one of a tank heat-exchanger, a tubular heat-exchanger, or a plate heat-exchanger.

14. A system according to claim 10, wherein the pressure in the high-pressure homogenizer is from 800 bars up to 2000 bars or preferably from 900 bars up to 1000 bars.

15. A system according to claim 10, wherein the separator is selected to be at least one of a centrifugal separator, a filter.

16. A system according to claim 11, wherein the dryer is selected to be at least one of a drum dryer or a spray dryer.

17. A system according to claim 10, wherein the microbial cells comprise isolated bacterial strain deposited as VTT-E-193585 or a derivative thereof.
